(19) United States
(12) Patent Application Publication
(10) Pub. No.: US 2025/0083306 A1
(43) Pub. Date: Mar. 13, 2025

(54) Graphene-Ni-C Coated TCP: Fabrication of Graphene-C-Ni-PVA Coated Mandrel-Coiled Twisted and Coiled Polymer Fishing Line (TCPFL) for Enhanced Performance

(71) Applicant: Toyota Motor Engineering & Manufacturing North America, Inc., Plano, TX (US)

(72) Inventors: Pawandeep Singh MATHARU, Dallas, TX (US); Yonas TADESSE, Garland, TX (US); Yuyang SONG, Ann Arbor, MI (US); Umesh GANDHI, Farmington Hills, MI (US)

(73) Assignees: Toyota Motor Engineering & Manufacturing North America, Inc., Plano, TX (US); The University of Texas at Dallas, Richardson, TX (US); Toyota Jidosha Kabushiki Kaisha, Aichi-ken (JP)

(21) Appl. No.: 18/463,832

(22) Filed: Sep. 8, 2023

(51) Int. Cl. B25J 9/10; D07B 1/16

(52) U.S. Cl. CPC B25J 9/1075; A61K 9/00

(57) ABSTRACT

A nanomaterial-coated mandrel-coiled twisted and coiled polymer fishing line (TCP_{FL}) actuator, method, and computer program product for creating the mandrel-coiled TCP_{FL}) actuator. A plurality of mandrel-coiled TCP_{FL} muscles may be fabricated, wherein fabricating the plurality of mandrel-coiled TCP_{FL} muscles may include synthesizing Graphene-C-Ni-PVA solution and coating the TCP_{FL} with the Graphene-C-Ni-PVA solution.

### BACKGROUND

Soft artificial muscles may be used for advanced applications like soft robots (both in air and underwater), orthotics, prosthetics, humanoid robots, wearables, medical devices, etc. This is because traditional actuators like motors and pumps are bulky, noisy and rigid.

### SUMMARY

In one example implementation, a method for creating a nanomaterial coated, mandrel-coiled twisted and coiled polymer fishing line (TCP_{FL}) actuator may include but is not limited to fabricating a plurality of mandrel-coiled TCP_{FL} muscles. Fabricating the plurality of mandrel-coiled TCP_{FL} muscles may include synthesizing Graphene-C-Ni-PVA solution and coating the TCP_{FL} with the Graphene-C-Ni-PVA solution.

One or more of the following example features may be included. Fabricating the plurality of mandrel-coiled TCP_{FL} muscles may further include twisting of polymer fibers. Fabricating the plurality of mandrel-coiled TCP_{FL} muscles may further include into the plurality of mandrel-coiled TCP_{FL} muscles. Fabricating the plurality of mandrel-coiled TCP_{FL} muscles may further include utilizing a mandrel coiling process. Fabricating the plurality of mandrel-coiled TCP_{FL} muscles may further include utilizing a thermal annealing process. Synthesizing the Graphene-C-Ni-PVA solution may include dissolving an amount of PVA in water to create a solution, adding Graphene powder, mesoporous C, and Ni nanoparticles to the solution, and removing supernatant from the solution. Coating the TCP_{FL} with the Graphene-C-Ni-PVA solution may include placing the TCP_{FL} in the solution, shaking the TCP_{FL} in the solution, drying the TCP_{FL} by placing the TCP_{FL} in a pre-heated environment for a predetermined amount of time, and crimping the TCP_{FL}.

In another example implementation, a computer program product may reside on a computer readable storage medium having a plurality of instructions stored thereon which, when executed across one or more processors, may cause at least a portion of the one or more processors to perform operations for creating a nanomaterial coated, mandrel-coiled twisted and coiled polymer fishing line (TCP_{FL}) actuator that may include but are not limited to fabricating a plurality of mandrel-coiled TCP_{FL} muscles. Fabricating the plurality of mandrel-coiled TCP_{FL} muscles may include synthesizing Graphene-C-Ni-PVA solution and coating the TCP_{FL} with the Graphene-C-Ni-PVA solution.

One or more of the following example features may be included. Fabricating the plurality of mandrel-coiled TCP_{FL} muscles may further include twisting of polymer fibers. Fabricating the plurality of mandrel-coiled TCP_{FL} muscles may further include into the plurality of mandrel-coiled TCP_{FL} muscles. Fabricating the plurality of mandrel-coiled TCP_{FL} muscles may further include utilizing a mandrel coiling process. Fabricating the plurality of mandrel-coiled TCP_{FL} muscles may further include utilizing a thermal annealing process. Synthesizing the Graphene-C-Ni-PVA solution may include dissolving an amount of PVA in water to create a solution, adding Graphene powder, mesoporous C, and Ni nanoparticles to the solution, and removing supernatant from the solution. Coating the TCP_{FL} with the Graphene-C-Ni-PVA solution may include placing the TCP_{FL} in the solution, shaking the TCP_{FL} in the solution, drying the TCP_{FL} by placing the TCP_{FL} in a pre-heated environment for a predetermined amount of time, and crimping the TCP_{FL}.

In another example implementation, a nanomaterial coated, mandrel-coiled twisted and coiled polymer fishing line (TCP_{FL}) actuator may include but is not limited to a plurality of mandrel-coiled TCP_{FL} muscles, wherein the plurality of mandrel-coiled TCP_{FL} muscles may be fabricated, wherein the plurality of mandrel-coiled TCP_{FL} muscles may be coated with a synthesized Graphene-C-Ni-PVA solution.

One or more of the following example features may be included. Fabricating the plurality of mandrel-coiled TCP_{FL} muscles may further include twisting of polymer fibers. Fabricating the plurality of mandrel-coiled TCP_{FL} muscles may further include into the plurality of mandrel-coiled TCP_{FL} muscles. Fabricating the plurality of mandrel-coiled TCP_{FL} muscles may further include utilizing a mandrel coiling process. Fabricating the plurality of mandrel-coiled TCP_{FL} muscles may further include utilizing a thermal annealing process. Synthesizing the Graphene-C-Ni-PVA solution may include dissolving an amount of PVA in water to create a solution, adding Graphene powder, mesoporous C, and Ni nanoparticles to the solution, and removing supernatant from the solution. Coating the TCP_{FL} with the Graphene-C-Ni-PVA solution may include placing the TCP_{FL} in the solution, shaking the TCP_{FL} in the solution, drying the TCP_{FL} by placing the TCP_{FL} in a pre-heated environment for a predetermined amount of time, and crimping the TCP_{FL}.

The details of one or more example implementations are set forth in the accompanying drawings and the description below. Other possible example features and/or possible example advantages will become apparent from the description, the drawings, and the claims. Some implementations may not have those possible example features and/or possible example advantages, and such possible example features and/or possible example advantages may not necessarily be required of some implementations.

### BRIEF DESCRIPTION OF DRAWINGS

FIG. 1 is an example diagrammatic view of an example Graphene-C-Ni-PVA coated mandrel coiled TCP_{FL} according to one or more example implementations of the disclosure;

FIG. 2 is an example diagrammatic view of an example experimental setup to create the mandrel-coiled TCP_{FL} muscles according to one or more example implementations of the disclosure;

FIG. 3 is an example diagrammatic view of a synthesis of Graphene-C-Ni-PVA nanomaterial solution preparation according to one or more example implementations of the disclosure;

FIG. 4 is an example diagrammatic view of a coating process according to one or more example implementations of the disclosure;

FIG. 5 is an example schematic diagram of a characteristic setup with a power supply, temperature sensor and the muscle according to one or more example implementations of the disclosure;

FIG. 6 is an example diagrammatic view of a displacement and actuation strain characterization according to one or more example implementations of the disclosure;

FIG. 7 is an example diagrammatic view of a comparison of dynamic performance according to one or more example implementations of the disclosure;

FIG. 8 is an example diagrammatic view of a comparison of power consumed (w) vs Time(s) for coated & non-coated actuator according to one or more example implementations of the disclosure;

FIG. 9 is an example diagrammatic view of a variation of temperature with time according to one or more example implementations of the disclosure;

FIG. 10 is an example diagrammatic view of a plot showing improvement in power consumed in percentage of watts according to one or more example implementations of the disclosure; and

FIG. 11 is an example flowchart of a fabrication process according to one or more example implementations of the disclosure.

Like reference symbols in the various drawings may indicate like elements.

### DESCRIPTION

Soft artificial muscles may be used for advanced applications like soft robots (both in air and underwater), orthotics, prosthetics, humanoid robots, wearables, medical devices, etc. This is because traditional actuators like motors and pumps are bulky, noisy and rigid. Hence, it may be beneficial to develop muscle-like actuators that are efficient, have high strain capabilities, high load carrying capacity, dynamic mechanical compliance, and/or high specific energy/power. Most of the metrics depend on the input driver of the actuator. Many actuators are presented like shape memory alloys, cavatappi (pneumatically or hydraulically driven, but in the shape of TCP), pneumatic actuators, electromagnetic actuators, HASEL actuator, thermally actuated twisted and coiled actuators (TCPs), etc.

To help reduce the power input of thermal muscles, an electrothermally actuated shape memory alloy (SMA) wires for an underwater jellyfish application have been tried. Conduits were provided that house the actuators to prevent heating all the surrounding water. Incorporating a nichrome resistance wire of smaller diameter (80 μm) onto the twisted fishing line, reduces the amount of power required to actuate the muscles. However, the actuator fabrication success rate is <50% when another person followed this approach, the load carrying capacity range shown for this actuator is also less (120 g), and the lifecycle shown was up to ˜2400 cycles (did not fail until this cycle).

The energy conversion efficiency of thermal actuation of coiled polymer fiber tensile actuators is generally very low (less than ˜0.5%). This problem has not been solved for TCP actuators to date. There is the potential application of self-coiled TCP fishing line actuators in underwater soft robotic applications, but, as there is no hydrophobic layer between the actuator and surrounding water, the input power provided to the actuators is all drained out in water. Hence, the power required is very high (>100 W) to make the soft robot swim in water. To overcome these drawbacks, it may be possible to wrap the actuators with Teflon tape to act as a layer between water and actuator.

There may be an improvement in dynamic behavior of thermally operated Twisted and coiled actuators (TCAs) by spray-coating graphene and silver nanoflowers (AgNFs) onto the surface of the actuator to achieve enhanced heat transfer. This approach used nylon sewing threads, which is different from nylon fishing lines, and did not show reduction in power consumed in the results.

TCP actuators may be developed from fishing line that lift 100 times heavier objects than a human muscle with a contractile strain of 49%. This may demonstrate an efficiency of ˜1%, as they wrapped CNT sheets over polyethylene fiber. Generally, thermally-driven actuators are less efficient and consume high power. The reason for this is the low thermal conductivity of polymer fibers. TCPs operate on the principle of Joule heating when an electric current is applied to it. Since the polymer fibers have low thermal conductivity, CNTs have been used to fabricate coiled artificial muscles, which provide large forces with fast tensile actuation, but are difficult to fabricate with technical complexities and high manufacturing costs. Nanomaterial coating with graphene flakes on silver-coated nylon fibers (nylon 6, 6 sewing threads, 260151023534, Shieldex) was introduced to enhance dynamic performance and take advantage of inexpensive polymer fibers. Later, to improve the cycle performance of these actuators there may be spray-coating of the TCP_{Ag} polymer fibers with Graphene/silver nanoflower hybrid solution. This demonstrated a reduction in total actuation cycle time by 38% and threefold larger cyclic peak-to-peak amplitude of the displacement than non-coated TCP_{Ag}. TCP_{Ag} refers to Twisted and Coiled Polymer with silver (Ag) coating. The precursor material is a multifilament nylon thread pre-coated with silver and available in the market.

Graphene has a unique 2D geometry that originates the unusual thermal properties providing a precursor to novel discoveries of heat-flow physics aiding in new thermal management applications. The thermal conductivity of graphene (having a perfect structure) is almost at par with diamond materials (>5000 W/mK). Graphene also has super-high aspect ratio and a 2D structural morphology, making it a suitable material as a filler for polymers in order to achieve very high thermal conductivity. Monolayer graphene having minimal defects illustrated a very high thermal conductivity of 2000 W/mK. Hence, in order to improve the thermal conductivity of TCP_{FL} artificial muscles, which will aid in faster dynamic actuation at lesser power consumption (enhanced efficiency), the present disclosure includes an example solution consisting of graphene powder (e.g., electrical conductivity >103 S/m), mesoporous C and Ni nanoparticles.

“Mesoporous carbon” generally refers to solid-based material, according to International Union of Pure and Applied Chemistry (IUPAC). They either have ordered or disordered networks with broad or narrow pores distributed in the range of, e.g., 2 to 50 nm. Mesoporous carbon provides good thermostability, high surface area, and large pore-volume, improving its functionality in various applications. Mesopores in carbon improved upon limitations like poor conductivity, structural integrity, and mass transport. Mesoporous carbon materials have extensive potential applications ranging from electrochemistry, energy storage, separation and adsorption, catalysis, etc. It may be demonstrated the used of metallic nanoparticles to improve the thermal conductivity by bridging two-dimensional (2D) materials. The present disclosure may use mesoporous carbon for fabrication of conductive filaments. It may be demonstrated the use of Ag/Ni metal mesh as transparent conductive electrode for optoelectronic applications.

The fifth-most abundant metal available on earth, Nickel, is used in applications for stabilizing other metals and strengthening. As is the case with cobalt, nickel can withstand high temperatures apart from being very strong and corrosion resistant. Nickel has very good thermal conductivity (91 W/mK) and electrical conductivity (14.3×106 S/m) with a melting temperature of 1728 K. Hence, it has been discovered to be a great material to stabilize and strengthen the nanomaterial coating solution for the TCP_{FL} artificial muscles. It has great potential in enhancing thermal conductivity of the low conductivity polymer of TCP actuators fabricated from monofilament fishing lines. Polyvinyl alcohol (PVA) is generally a biocompatible, highly hydrophilic polymer with nontoxic, and has excellent film-forming properties. It also has clean burning characteristics and excellent binding strength. Hence, PVA may be utilized as the binding material for the mesoporous carbon-NiAg metal mesh coating solution.

As will be discussed in greater detail below, the present disclosure may embed Ni nanoparticles into Graphene powder and mesoporous carbon with PVA working as the binding material to enhance thermal contacts, thus improving the dynamic performance of TCP_{FL}. The interfaces between Graphene powder and mesoporous C may be bridged by the Nickel nanoparticles at ˜80° C., ensuring enhanced thermal contacts. One-pot magnetic stirrer process may be utilized to synthesize the Graphene-C-Ni-PVA solution that is coated onto the mandrel-coiled TCP_{FL} by manual (or automated) shaking. The enhanced isotonic performance of the Graphene-C-Ni-PVA coated mandrel-coiled TCP_{FL} (Graph-TCP) was investigated by cyclic operations by providing input power.

A nanomaterial-coated, mandrel-coiled twisted and coiled polymer fishing line actuator (e.g., with nichrome untwisted wire (TCP_{FL}^{NMC})), method, and computer program product for creating the mandrel-coiled TCP_{FL}^{NMC} actuator. A plurality of mandrel-coiled TCP_{FL}^{NMC}_{L} muscles may be fabricated, wherein fabricating the plurality of mandrel-coiled TCP_{FL}^{NMC} muscles may include synthesizing Graphene-C-Ni-PVAGraphene-C-Ni-PVA solution and coating the TCP_{FL}^{NMC} with the Graphene-C-Ni-PVA solution.

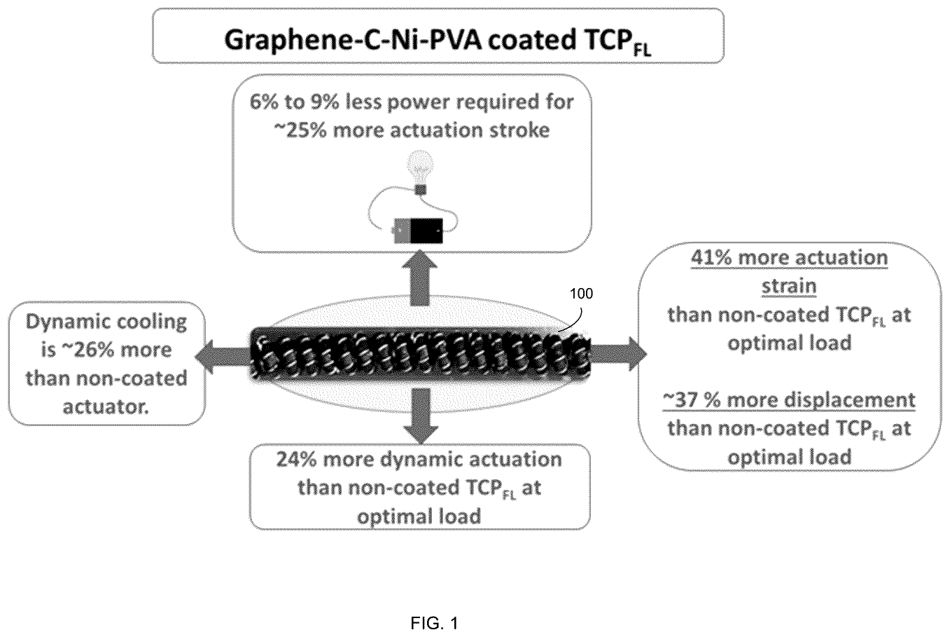

As can be seen from the example FIG. 1, an example coil (e.g., a Graphene-C-Ni-PVA coated mandrel coiled TCP_{FL}) 100 is shown. As can be seen, example and non-limiting advantages of the present disclosure may include, e.g.:

Actuation strain: Graphene-C-Ni-PVA coated mandrel coiled TCP_{FL} with, e.g., ˜41% more actuation strain and ˜37.5% more actuation displacement than similar length (e.g., 60 mm unloaded, 69 mm loaded for 70 g pre-stress weight) non-coated TCP_{FL} at 0.27 A input currents condition.

Dynamic Actuation: This nanomaterial coated mandrel coiled TCP_{FL} may improve the dynamic actuation by, e.g., ˜24%, ˜36% and ˜11% for pre-stress loads of, e.g., 70 g, 100 g and 150 g respectively, clearly showing the advantages of using the Graphene-C-Ni-PVA solution.

Cooling rate: Nanomaterial coated mandrel coiled TCP_{FL} may improve the cooling rate of the actuator by, e.g., ˜26%, ˜54% and ˜11% for pre-stress loads of, e.g., 70 g, 100 g and 150 g respectively.

Power consumption: For similar input conditions for current (e.g., 0.25 A, 0.27 A, 0.29 A), may show less power consumption ranging from, e.g., ˜6% to ˜9%, while providing, e.g., 25% more actuation strain on average.

Thus, the present disclosure may include twist-inserted, nichrome (or other material) incorporated, coiled mandrel-coiled, “Graphene-C-Ni-Polyvinyl Alcohol (PVA) coated” nylon fishing line fibers that serve as artificial muscles to produce tensile actuation by taking comparatively lower power than non-coated artificial muscles of similar type. These actuators (e.g., nanomaterial-coated mandrel-coiled twisted and coiled polymer fishing line actuator with nichrome untwisted wire (TCP_{FL}^{NMC}) generate more tensile actuation when powered electrically. These artificial muscles utilize twisted and coiled fibers and include a coating on the outer surface.

“Graphene-C-Ni-PVA” may generally include Graphene, Mesoporous Carbon, Nickel Silver and Polyvinyl Alcohol. The present disclosure may enable the TCP (twisted and coiled polymer) fishing line actuators to operate at lower input power and generate more tensile actuation than non-coated TCP. Graphene-C-Ni-PVA coating on TCP fishing line may demonstrate unique actuators that reduce the power requirements by, e.g., ˜6 to 9% from conventional TCP fishing line actuators. As will be discussed below, the present disclosure may introduce a Graphene powder with mesoporous Carbon nano powder and Ni nanoparticle-PVA mixture. Mesoporous Carbon (also known as carbon Black, CB) has high surface area, large pore-volume, good thermostability and can act as catalytic support. Graphene also has super-high aspect ratio and a 2D structural morphology, making it a suitable material as a filler for polymers in order to achieve very high thermal conductivity. Mesoporous Carbon is hydrophobic to water, and this allows the coating to create a layer between the actuator and surrounding water, which will lead to power saving if applied in underwater applications. In some implementations, nickel nanoparticles may be utilized to these unique Graphene-C-Ni-PVA coated TCP fishing line actuators to achieve higher thermal conductivity, improving the dynamic behavior and resulting actuation frequency (e.g., ˜20% to ˜41% more actuation strain than non-coated actuators). The Graphene-C-Ni-PVA coating may improve the thermal conductivity and cooling rate of the TCP fishing line actuator, as will be discussed below.

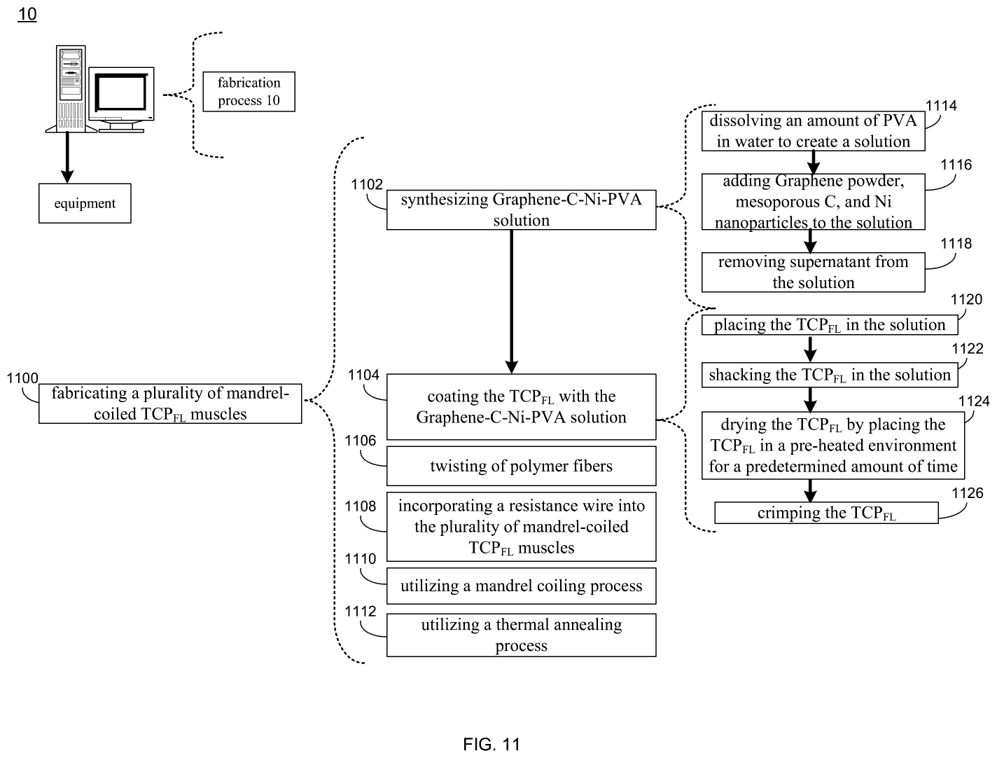

As discussed above and referring also at least to the example implementations of FIGS. 1-11, fabrication process 10 (as shown in FIG. 11) may create a mandrel-coiled twisted and coiled polymer fishing line (TCP_{FL}) actuator, which may include but is not limited to fabricating 1100 a plurality of mandrel-coiled TCP_{FL} muscles. Fabricating the plurality of mandrel-coiled TCP_{FL} muscles may include fabrication process 10 synthesizing 1102 Graphene-C-Ni-PVA solution. Fabricating the plurality of mandrel-coiled TCP_{FL} muscles may include fabrication process 10 coating 1104 the TCP_{FL} with the Graphene-C-Ni-PVA solution.

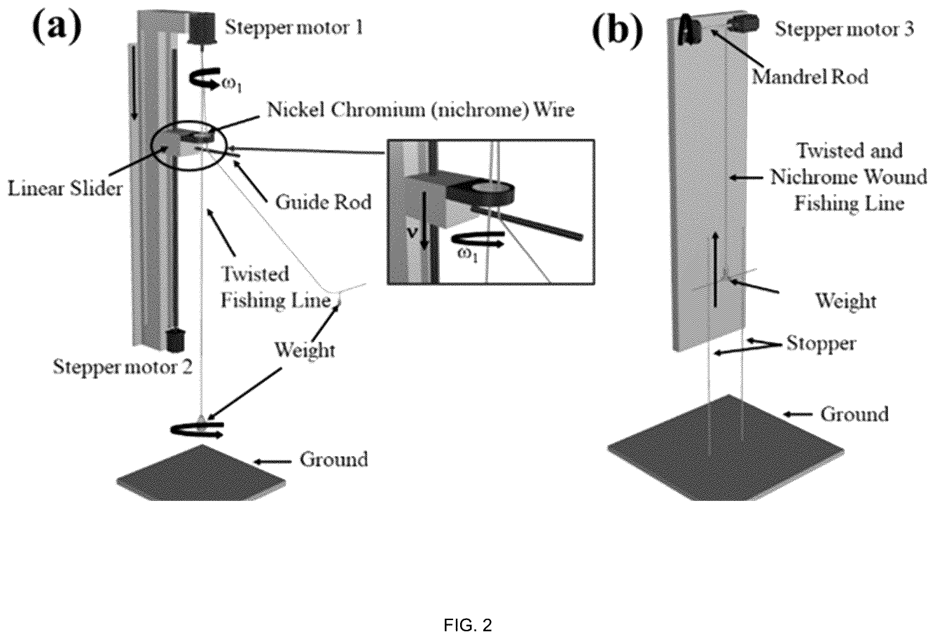

In some implementations, fabrication process 10 may create a mandrel-coiled twisted and coiled polymer fishing line (TCP_{FL}) actuator, which may include fabricating 1100 a plurality of mandrel-coiled TCP_{FL} muscles. For instance, the line (e.g., fishing line) may be a non-conductive material, and the incorporation of a heating wire (e.g., a nichrome heating wire) may be used to effectively heat the precursor material. This provides convenient Joule heating for electrothermal actuation. An example experimental setup 200 is shown in FIG. 2(a-b) to create the mandrel-coiled TCP_{FL} muscles with the help of mandrel (e.g., 1.4 mm diameter) coiling. Other sizes may be used without departing from the scope of the present disclosure. The example nichrome wire (e.g., 160 μm diameter) may be used as the heating element for the monofilament fishing line (e.g., 0.8 mm diameter). The challenge of easily wrapping a thin nichrome wire may be addressed by the example custom fabrication setup shown in FIG. 2. Different steps of the fabrication process are described below.

In some implementations, fabricating 1100 the plurality of mandrel-coiled TCP_{FL} muscles may further include twisting 1106 of polymer fibers but not in the resistance wire. For instance, a certain fishing line length (e.g., 1200 mm) may be cut in order to fit a linear motion slider travelling range. Both the ends of the fishing line may be tied with safety pins (or other fastener); one end may be connected to the motor shaft (as shown in FIG. 2(a)). A deadweight of, e.g., 500 g may be connected to the bottom end of the fishing line with a stopper shaft in order to incorporate the twist. The motor may be rotated at a speed of, e.g., 300 rpm in a counterclockwise (or clockwise) direction. After a few minutes, there may be a twist inserted in the fiber as it shrinks in length and the 500 g load may gently start to move up. As the first coiling in the fiber is noticed (or after a shrinkage of a predefined length), the motor may be stopped, and the twist insertion may be concluded.

In some implementations, fabricating 1100 the plurality of mandrel-coiled TCP_{FL} muscles may further include incorporating 1108 a resistance wire (untwisted) into the plurality of mandrel-coiled TCP_{FL} muscles. For instance, the example nichrome wire incorporation may be the next step in the fabrication process as shown in FIG. 2(a). Firstly, the stopper used in the previous step may be removed from the bottom end of the twist inserted fishing line. A new untwisted fishing line with the 500 g weight may be attached to the bottom end of the twist inserted fishing line so that the weight hanging just touched the ground and freely rotated. The nichrome wire may be attached to the top end and both the nichrome wire, and the twist inserted fishing line may be placed within the guide carriage and guide rod as shown in FIG. 2(a). Both the motors (1 and 2) may be started at different speeds (e.g., Motor 1—300 rpm, Motor 2—150 rpm) until the nichrome incorporation was done for the full length of the fishing line. At these speeds, the pitch of the nichrome incorporated fiber may be approximately 0.42 mm. While the resistance wire incorporation takes place, the wire may be guided around the guide rod and a 50 g weight may be hung to keep it taut during the wire incorporation process. This may help in maintaining a constant pitch and wrapping angle.

In some implementations, fabricating 1100 the plurality of mandrel-coiled TCP_{FL} muscles may further include utilizing 1110 a mandrel coiling process. For instance, FIG. 2(b) shows the mandrel coiling process for the nichrome wrapped twist inserted fiber. A safety pin (or other fastener) from the top end may be attached with the stepper motor-3 coupling and the 500 g weight may be replaced by a, e.g., 200 g weight in order to make it easier for the stepper motor to coil the fiber. A mandrel diameter of, e.g., 1.4 mm may be chosen for example purposes only, and the stepper motor may be started at a speed of, e.g., 300 rpm in counterclockwise (or clockwise) direction. This may allow the fiber to coil and allow the finished product to be homochiral in nature; while if the coiling had been done in the opposite direction to which the fiber was twisted, it would have resulted in heterochiral muscle.

In some implementations, fabricating 1100 the plurality of mandrel-coiled TCP_{FL} muscles may further include utilizing 1112 a thermal annealing process. Fr instance, the final step of the mandrel coiled TCP_{FL} muscle fabrication process may be the muscle annealing, which may be done in a furnace or the like. The mandrel coiled fiber along with a mandrel rod may be put inside the furnace, pre-heated at a temperature of, e.g., 180° C. To keep the muscle shape and pitch intact, both the ends may be fixed to clamps on a metal plate and the whole frame may be placed inside the preheated furnace for, e.g., 80 minutes. In some implementations, the muscle may be inverted after 40 minutes (half the total time) to have uniform heating throughout. It will be appreciated that various other temperatures and timing may be used without departing from the scope of the present disclosure.

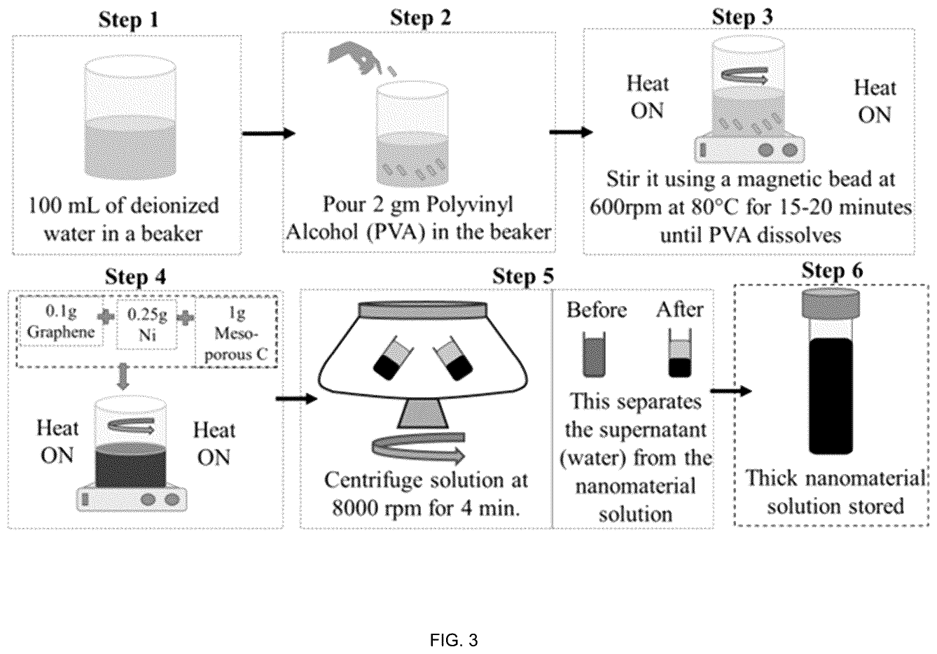

In some implementations, fabricating 1100 the plurality of mandrel-coiled TCP_{FL} muscles may include fabrication process 10 synthesizing 1102 Graphene-C-Ni-PVA solution, and in some implementations, synthesizing 1102 the Graphene-C-Ni-PVA solution may include dissolving 1114 an amount of PVA in water to create a solution, adding 1116 Graphene powder, mesoporous C, and Ni nanoparticles to the solution, and removing 1118 supernatant from the solution. For instance, an example nanomaterial solution preparation 300 (e.g., synthesis of Graphene-C-Ni-PVA solution) is shown in FIG. 3.

As can be seen in FIG. 3 (step 1), an amount of distilled water (e.g., 100 ml of distilled water) may be measured into a, e.g., 600 ml glass beaker. Then, 2 gm of polyvinyl alcohol (PVA) may be added to the distilled water (Step 2), and the beaker may be placed onto a stirrer (e.g., a magnetic stirrer) with a magnetic bead at, e.g., 800 rpm and, e.g., 75-80° C. for, e.g., ˜15-20 minutes or until the PVA is fully dissolved in water to give a clear solution (Step 3). After this, 0.1 gm of Graphene powder, 1 gm of mesoporous C and 0.25 g of Ni nanoparticles may be added to the solution, that is stirred at, e.g., 600 rpm and, e.g., 50° C. for, e.g., ˜3 hours (Step 4). After observing the solubility of the black solution, heat may be turned off and the reaction material may be allowed to cool down keeping the stirring on at, e.g., 600 rpm. The cooled down solution may then be centrifuged at, e.g., 8000 rpm for, e.g., ˜4 to 5 minutes (Step 5), so that excess water (supernatant) separates out from the thick solution. This supernatant may be kept in a clean beaker and some of it may be added to the thick solution as required to re-dissolve. This may be the final synthesized solution for coating the actuator. It will be appreciated that the variables described throughout may vary depending on the scale of creation desired. As such, the size, amounts, timing, speed, etc. described throughout should be taken as example only and not to otherwise limit the scope of the present disclosure.

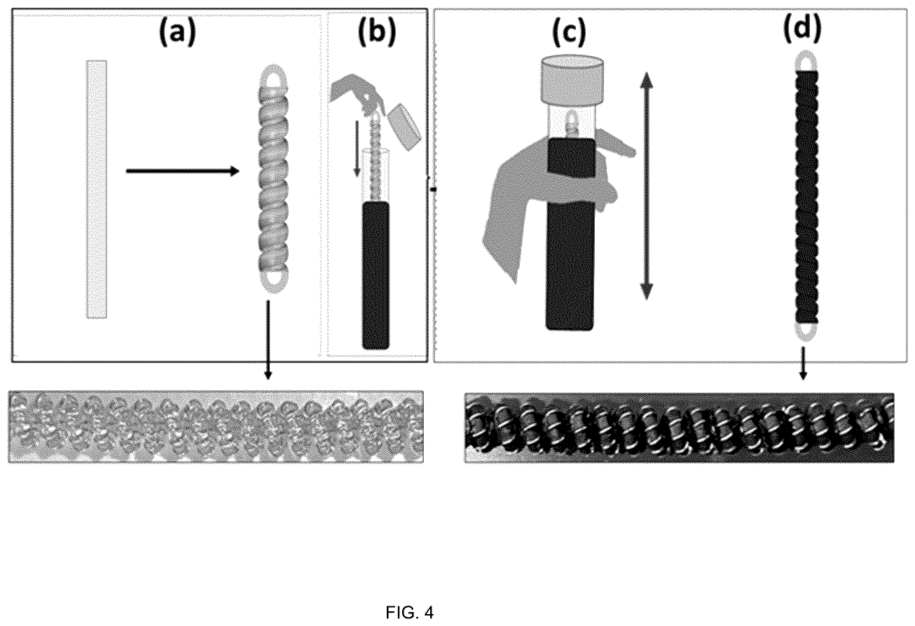

In some implementations, fabricating 1100 the plurality of mandrel-coiled TCP_{FL} muscles may include fabrication process 10 coating 1104 the TCP_{FL} with the Graphene-C-Ni-PVA solution, and in some implementations, coating 1104 the TCP_{FL} with the Graphene-C-Ni-PVA solution may include placing 1120 the TCP_{FL} in the solution, shaking 1122 the TCP_{FL} in the solution, drying 1124 the TCP_{FL} by placing the TCP_{FL} in a pre-heated environment for a predetermined amount of time, and crimping 1126 the TCP_{FL}. For instance, once mandrel coiled TCP_{FL} is fabricated, and Graphene-C-Ni-PVA solution is synthesized, the coating process may be undertaken. An example coating process 400 of TCP_{FL} actuator is shown in the example implementation of FIG. 4. In the example, the example 60 mm long (e.g., 3.4 mm diameter), non-coated TCP_{FL}, shown in FIG. 4(a) may be put into the nanomaterial solution inside a vial (shown in FIG. 4(b)). In some implementations, to make sure the actuator is as perfectly coated as possible, it may be rigorously shaken manually (or via machine) for, e.g., ˜2 minutes (shown in FIG. 4(c)). Once done, the wet actuator may be taken out of the vial and kept in a pre-heated oven at, e.g., 80° C. for, e.g., 60 minutes to dry (shown in FIG. 4(d)). The finished actuator may be taken out and crimped for use.

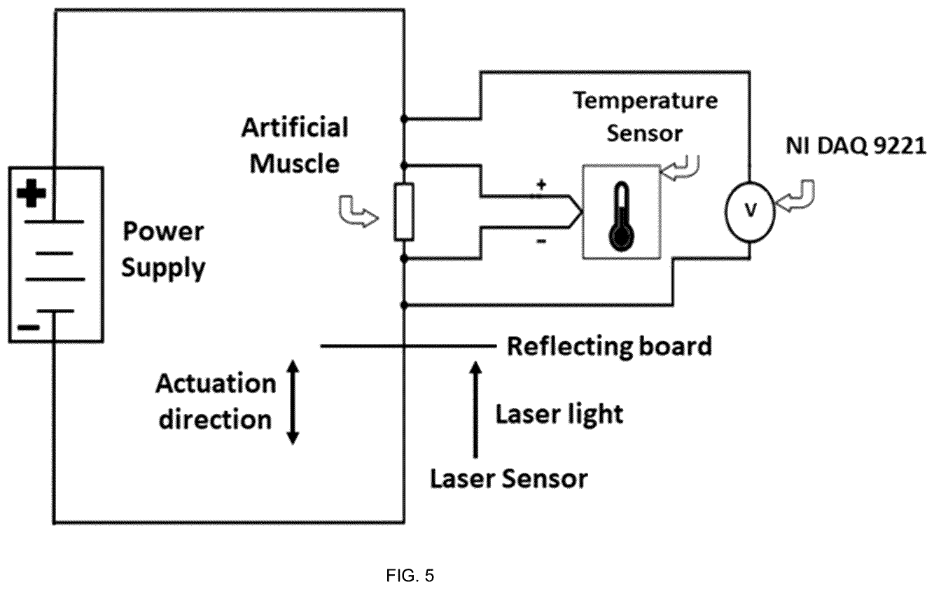

An example characterization setup was used to determine and compare the capabilities of the coated and non-coated TCP_{FL} muscles. This not only measured the temperature that a muscle was able to rise to over time, but also measured the voltage across the muscle, when a constant example current input was provided through BK Precision 9116 power source. FIG. 5 shows the example schematic diagram characteristic setup 500 with a power supply, temperature sensor and the muscle. A National Instruments DAQ 9221 module was connected in parallel with the muscle to measure the voltage across the muscle, as voltage stays equivalent across parallel circuits. At the center of both the actuators (coated and non-coated), a thermocouple was tied to the muscle to determine the temperature. A 70 g mass pulled on the muscle at one end, and at the opposing end. All of these measurements were sent to a computer for data collection using Lab View software. The characterization setup described in FIG. 5 was used to conduct isotonic tests between the coated and non-coated TCP_{FL} artificial muscles to determine the improvement in performance after coating the TCP_{FL}.

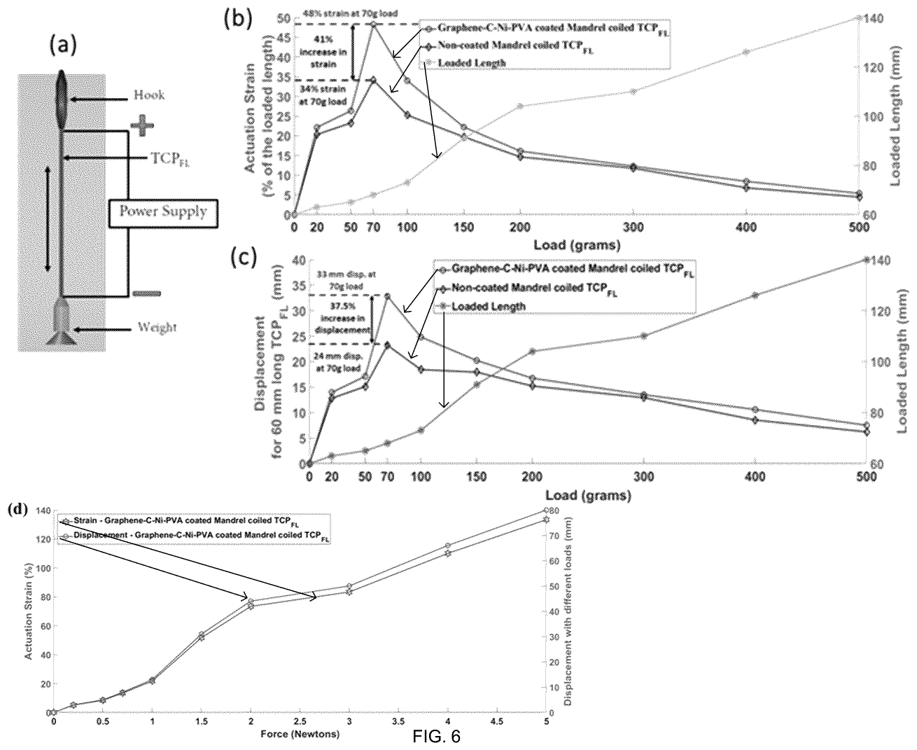

Displacement and actuation strain characterization are conducted by fixing the nanomaterial coated TCP_{FL} from a hook on one side and attaching different loads from the other side (shown in FIG. 6(a)). Actuator characterization was conducted to gain an insight into their mechanical performance for 15 s heating and 20 s cooling (0.0285 Hz actuation frequency and ˜43% duty cycle) at different loads until 500 g. The blocking force was determined and found to be 500 g where the muscles just break if input current is provided.

FIG. 6(b) shows the load dependence of percentage tensile strain at loaded length, while FIG. 6(c) illustrates the same for actuation displacement of 60 mm long TCP_{FL} (both coated and non-coated) at 0.0285 Hz (15 s heating, 20 s cooling) actuation frequency. The maximum displacement (˜33 mm) for Graphene-C-Ni-PVA coated TCP_{FL} actuator occurred at a constant 70 g load, while the maximum displacement (˜24 mm) for conventional TCP_{FL} actuator of similar unloaded length (60 mm) occurred at 70 g load. It is seen that the optimal range of actuation for these actuators is between 20 g to 100 g. The applied load is an important parameter as the tensile strain for loaded length of the muscle depends on the applied load as the actuator goes through considerable elongation under increasing load. The maximum percentage of actuation strain for a constant load of 70 g for Graphene-C-Ni-PVA coated TCP_{FL} actuator was noted to be ˜48% at an input current of 0.27 A, 15.8 V, 4.4 W (time period of 35 s and a duty cycle of ˜43%). This was 41% more than the actuation strain (˜14% difference) achieved for the conventional TCP_{FL} actuator (0.27 A, 17.4 V, 4.7 W). It is to be noted that both the types of actuators were powered at similar input power capacity. The loaded length of the TCP_{FL} actuator (FIG. 6(b)) is directly proportional to applied loads. Similarly, an improvement of ˜37.5% can be observed for the coated actuator at 70 g at similar input current (0.27 A) for both the actuators (coated and non-coated). FIG. 6. characterization comparison of 80 lb capacity fishing line (0.8 mm diameter) Graphene-C-Ni-PVA coated TCP_{FL} actuator and conventional TCP_{FL} actuator at 0.0285 Hz actuation frequency at different loads till 500 g. (a) Schematic diagram of training and characterization setup. (b) (Comparison of actuation strain with loaded length at different loads at similar input current for both actuators) Comparison of Actuation strain (% of the loaded length), vs different loads in grams of Graphene-C-Ni-PVA coated TCP_{FL} actuator (0.27 A, 15.8V) and conventional TCP_{FL}^{NMC} actuator (0.27 A, 17.4V) of 60 mm length each. (c) (Comparison of actuation displacement with loaded length at different loads at similar input current for both actuators) Comparison of y-axis displacement with loaded length (mm) vs different loads in grams of Graphene-C-Ni-PVA coated TCP_{FL} actuator (0.27 A, 15.8V) and conventional TCP_{FL}^{NMC} actuator (0.27 A, 17.4V) of 60 mm length each. (d)). Stiffness test results for Graphene-C-Ni-PVA coated TCP_{FL} actuator.

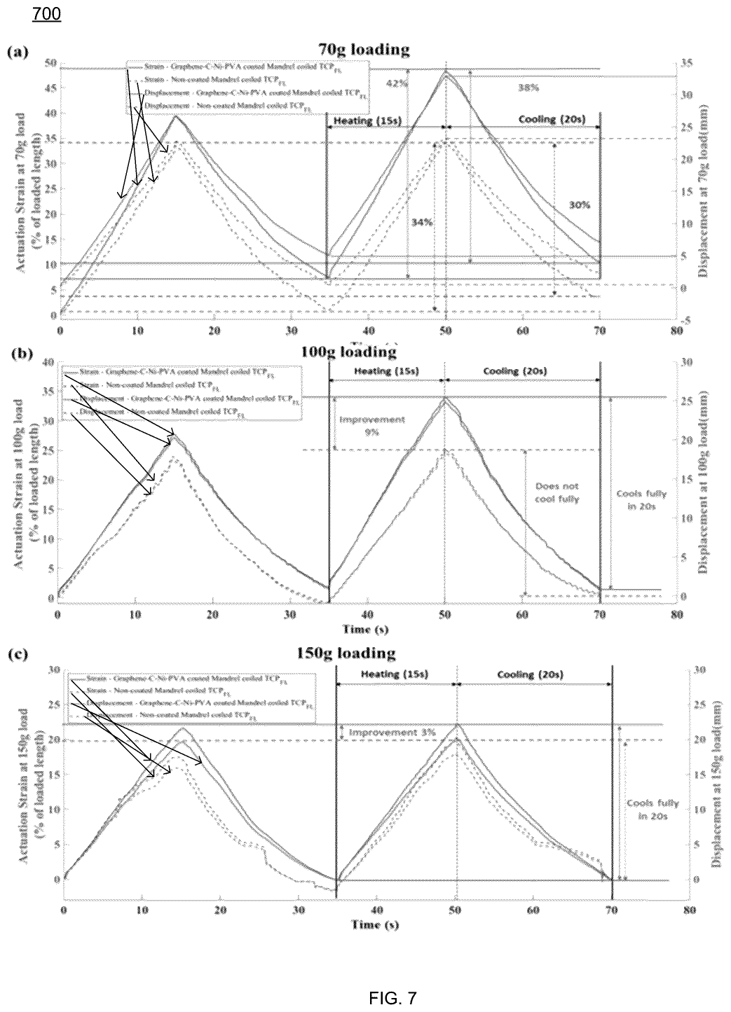

Also calculated was the actuation strain (% with loaded length) over time variation. The reason for this was to have a common metric for the same actuator of different lengths. In terms of actuation strain, which is a metric to measure the displacement performance for the same actuator with different lengths. FIG. 7(a) to (c) show the comparison of dynamic performance 700 of both types of actuators along with the faster heat dissipator between the two at different loadings. From these graphs, it can be seen the superior nature of Graphene-C-Ni-PVA coated TCP_{FL} actuator (powered at 4.3 W) over conventional TCP_{FL} actuator (powered at 4.7 W). The three figures show the tensile actuation, y-axis displacement vs time graphs at different loads (FIG. 7(a) for 70 g load, FIG. 7(b) for 100 g load, FIG. 7(c) for 150 g load).

It can be seen from the three graphs that the dynamic response of Graphene-C-Ni-PVA coated TCP_{FL} actuator is faster than the conventional TCP_{FL} actuator as it reaches the same tensile strain value as the conventional actuator, about 4 to 6 s faster (˜40% faster than total heating time of 15 s) for 70 g loading, while it is similar (5 to 6 s faster than total heating time of 15 s) for 100 g loading. For a heavier weight (150 g), the coated actuator can show similar actuation strain 2 to 3 s earlier (20% faster than total heating time of 15 s). For similar input current of 0.27 A, the coated actuator has ˜24%, ˜36% and ˜16% “more actuation strain” respectively than non-coated actuator. While the coated actuator cools ˜27%, ˜54% and ˜11% “more” at 70 g, 100 g and 150 g loading respectively. It is also observed that the coated actuator returns fully to the original position for 100 g and 150 g loading within 20 s of cooling time, however, the same cannot be said for the non-coated conventional TCP_{FL}.

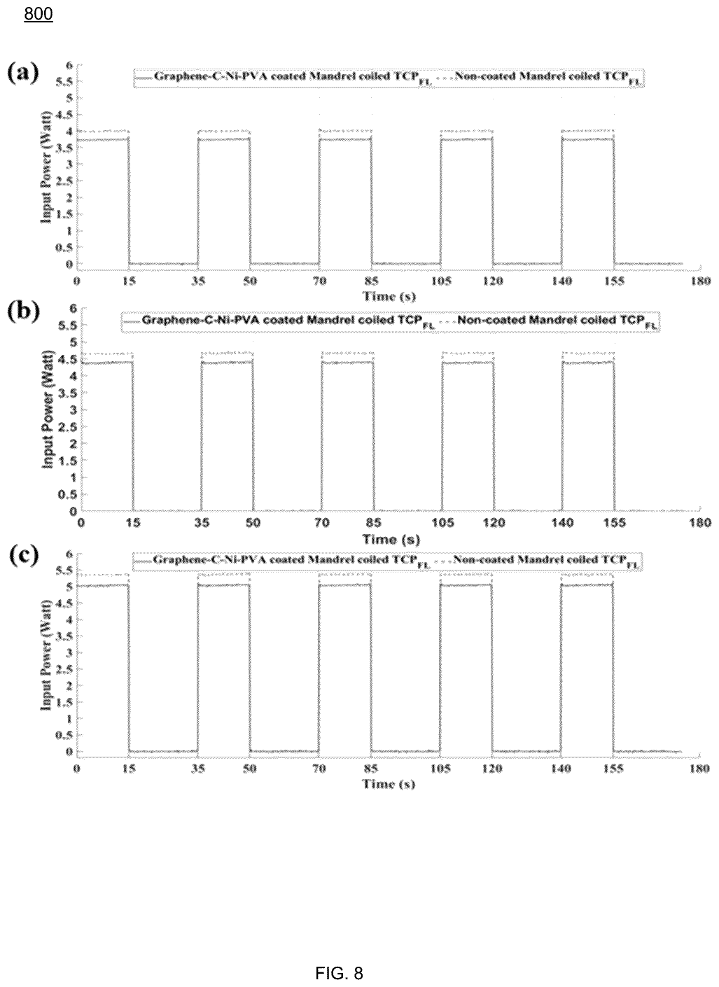

As shown in FIG. 8, there is a comparison 800 of power consumed (w) vs Time(s) for coated & non-coated actuator. Plots for coated and non-coated mandrel coiled TCP_{FL} at different input currents (a) 0.25 A, (b) 0.27 A, and (c) 0.29 A at 0.0285 Hz (15 s on, 20 s off) frequency.

Power consumption is an important factor that has to be improved for electrothermally actuated TCP actuators. FIG. 8(a) to (c) illustrates the improved power consumption from the output voltages noted from the isotonic tests for constant input currents (0.25 A, 0.27 A, 0.29 A) for both coated and non-coated actuators. At 0.25 A input current, the voltage and power consumed for the coated TCP_{FL} was ˜15 V (3.75 W), which is an improvement of ˜6% over the non-coated TCP_{FL} (16 V, 4 W). Again, at 0.27 A input current, the output voltage/power consumed for the coated TCP_{FL} was ˜15.9 V (4.3 W), which is a 8.5% (9%) improvement over non-coated TCP_{FL} (˜17.4 V, 4.7 W). At 0.29 A, we observed a ˜7.4% improvement in power consumption of coated TCP_{FL} (17.2 V, 5 W) to the non-coated TCP_{FL} (18.6 V, 5.4 W).

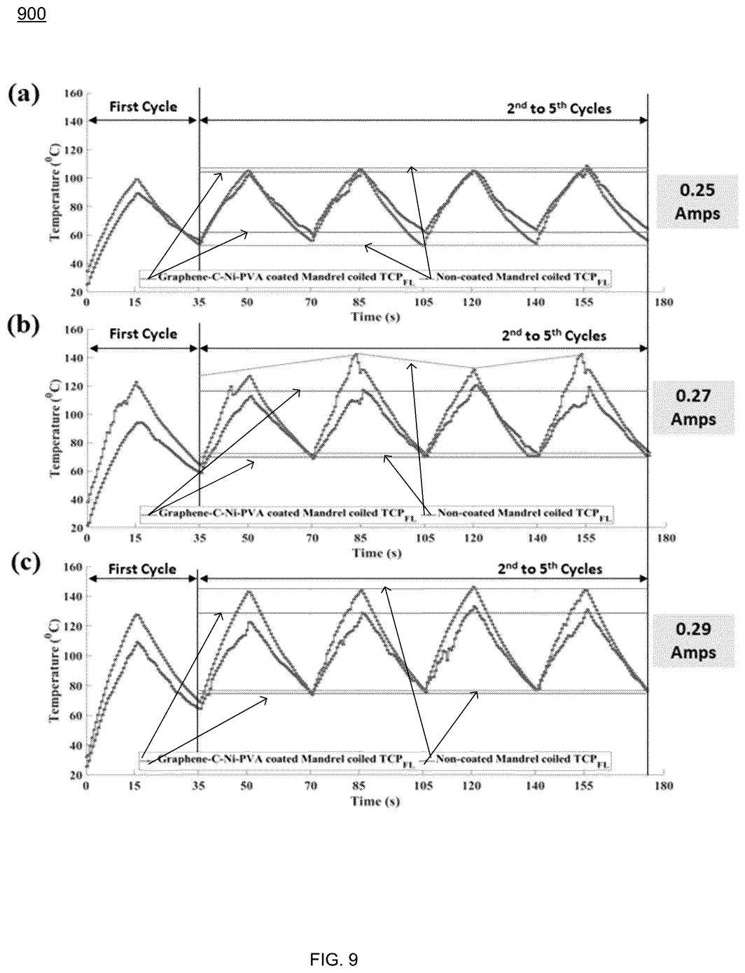

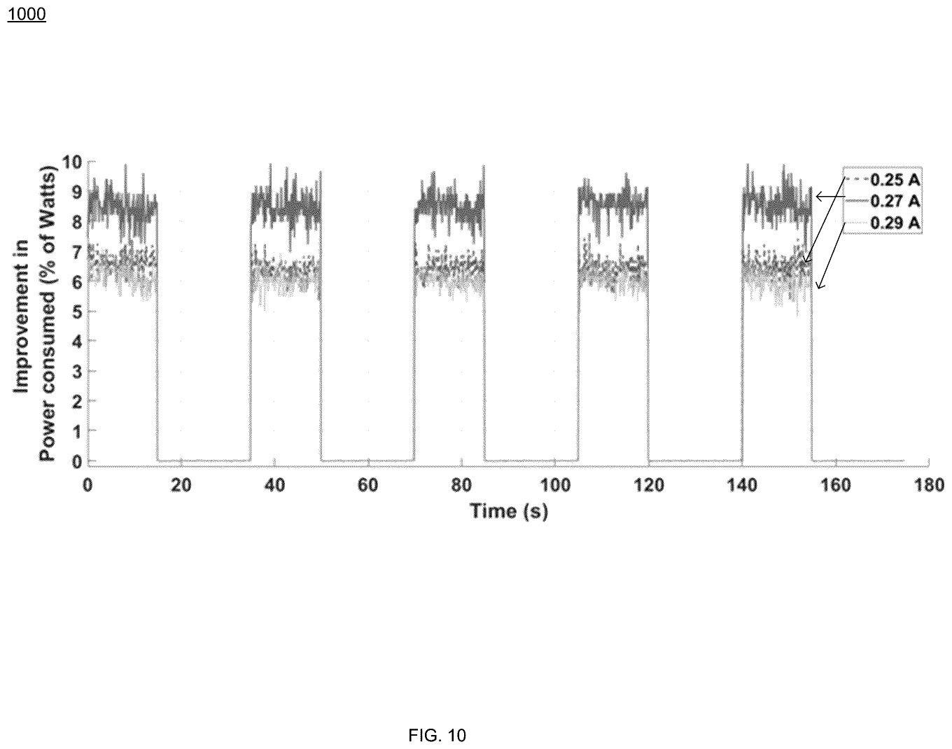

FIG. 9(a) to (c) illustrate variation of temperature with time 900 for the input currents of 0.25 A, 0.27 A and 0.29 A with a heating time of 15 s and cooling time of 20 s (0.0285 Hz) for coated & non-coated actuator. As the plots shown are for 1^{st} five cycles (1^{st}, 2^{nd}, 3^{rd}, 4^{th}, and 5^{th} cycles), it shows that the starting temperature for cycles other than the first cycle is higher than room temperature. This is because after the first cycle the actuator retains the heat for both the muscles (coated and non-coated). However, it is to be noted that the starting temperature of the 2^{nd} cycle is more for the non-coated actuator (˜57° C. for 0.25 A, ˜65° C. for 0.27 A and ˜70° C. for 0.29 A) than the coated actuator (57° C. for 0.25 A, ˜60° C. for 0.27 A and ˜65° C. for 0.29 A), proving that the coated actuator cools faster with nanomaterial coating. The figures also show that the coated actuator rises to lower temperature as the heat is distributed more uniformly throughout compared to a non-coated actuator. The plot 1000 in FIG. 10 shows the same variation, but as an improvement in power consumed in percentage of watts.

Thus, in some implementations, the dynamic behavior, actuation strain/displacement, and power consumption of mandrel-coiled TCP_{FL}, an electrothermal artificial muscle, were improved by treating the surface of the muscle with Graphene-C-Ni-PVA coating. In some implementations, a one-pot synthesis, utilizing a magnetic stirrer and centrifuge may be used to make this solution. A simple manual/automated shaking and annealing may be used to coat a dry, uniform layer of this material on the fabricated actuator of, e.g., diameter 3.4 mm and 60 mm long. Graphene powder and Ni nanoparticles may be coated with mesoporous carbon, while the PVA may be utilized as a binding agent. The coating on the TCP_{FL}, improved its thermal conductivity and natural actuation behavior.

The Graphene-C-Ni-PVA coating improved the actuation displacement by ˜18% to ˜36% (for different loads) while improving the actuation strain by at least ˜20% to ˜42% (for different loads) for input current of 0.27 A. It was observed from the experiments that actuation strain of these actuators is directly proportional to input power provided. The performance of the coated TCP_{FL} improved due to better thermal contacts of the coating, and hence improving the heat transfer to and from the actuator. Furthermore, the power consumed was ˜6% to ˜9% less than same actuator characteristics and same constant input current values.

It will be appreciated after reading the present disclosure that any standard fabrication equipment, as well as any other necessary equipment, may be used singly or in any combination with fabrication process 10, which may be operatively connected to a computing device, such as the computing device shown in FIG. 11, to obtain their instructions for creating one or more aspects of the present disclosure. In one or more example implementations, the respective flowcharts may be manually-implemented, computer-implemented, or a combination thereof. As will be appreciated by one skilled in the art after reading the present disclosure, the present disclosure may be embodied as a method, apparatus, or computer program product. Accordingly, the present disclosure may take the form of an entirely hardware implementation, an entirely software implementation (including firmware, resident software, micro-code, etc.) or an implementation combining software and hardware aspects that may all generally be referred to herein as a “circuit,” “module” or “system.” Furthermore, the present disclosure may take the form of a computer program product on a computer-usable storage medium having computer-usable program code embodied in the medium.

Any suitable computer usable or computer readable medium (or media) may be utilized. The computer readable medium may be a computer readable signal medium or a computer readable storage medium. The computer-usable, or computer-readable, storage medium (including a storage device associated with a computing device or client electronic device) may be, for example, but is not limited to, an electronic, magnetic, optical, electromagnetic, infrared, or semiconductor system, apparatus, device, or any suitable combination of the foregoing. More specific examples (a non-exhaustive list) of the computer-readable medium or storage device may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, solid state drives (SSDs), a digital versatile disk (DVD), a Blu-ray disc, and an Ultra HD Blu-ray disc, a static random access memory (SRAM), dynamic RAM (DRAM), synchronous DRAM (SDRAM), synchronous graphics RAM (SGRAM), and video RAM (VRAM), analog magnetic tape, digital magnetic tape, rotating hard disk drive (HDDs), a memory stick, a floppy disk, a mechanically encoded device such as punch-cards or raised structures in a groove having instructions recorded thereon, a media such as those supporting the internet or an intranet, or a magnetic storage device. Note that the computer-usable or computer-readable medium could even be a suitable medium upon which the program is stored, scanned, compiled, interpreted, or otherwise processed in a suitable manner, if necessary, and then stored in a computer memory. In the context of the present disclosure, a computer-usable or computer-readable, storage medium may be any tangible medium that can contain or store a program for use by or in connection with the instruction execution system, apparatus, or device.

Examples of storage implemented by the storage hardware include a distributed ledger, such as a permissioned or permissionless blockchain. Entities recording transactions, such as in a blockchain, may reach consensus using an algorithm such as proof-of-stake, proof-of-work, and proof-of-storage. Elements of the present disclosure may be represented by or encoded as non-fungible tokens (NFTs). Ownership rights related to the non-fungible tokens may be recorded in or referenced by a distributed ledger. Transactions initiated by or relevant to the present disclosure may use one or both of fiat currency and cryptocurrencies, examples of which include bitcoin and ether.

In some implementations, a computer readable signal medium may include a propagated data signal with computer readable program code embodied therein, for example, in baseband or as part of a carrier wave. In some implementations, such a propagated signal may take any of a variety of forms, including, but not limited to, electromagnetic, optical, or any suitable combination thereof. In some implementations, the computer readable program code may be transmitted using any appropriate medium, including but not limited to the internet, wireline, optical fiber cable, RF, etc. In some implementations, a computer readable signal medium may be any computer readable medium that is not a computer readable storage medium and that can communicate, propagate, or transport a program for use by or in connection with an instruction execution system, apparatus, or device.

In some implementations, computer program code for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java®, Smalltalk, C++ or the like. Java® and all Java-based trademarks and logos are trademarks or registered trademarks of Oracle and/or its affiliates. However, the computer program code for carrying out operations of the present disclosure may also be written in conventional procedural programming languages, such as the “C” programming language, PASCAL, or similar programming languages, as well as in scripting languages such as JavaScript, PERL, or Python. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a network, such as a cellular network, local area network (LAN), a wide area network (WAN), a body area network BAN), a personal area network (PAN), a metropolitan area network (MAN), etc., or the connection may be made to an external computer (for example, through the internet using an Internet Service Provider). The networks may include one or more of point-to-point and mesh technologies. Data transmitted or received by the networking components may traverse the same or different networks. Networks may be connected to each other over a WAN or point-to-point leased lines using technologies such as Multiprotocol Label Switching (MPLS) and virtual private networks (VPNs), etc. In some implementations, electronic circuitry including, for example, programmable logic circuitry, an application specific integrated circuit (ASIC), gate arrays such as field-programmable gate arrays (FPGAs) or other hardware accelerators, micro-controller units (MCUs), or programmable logic arrays (PLAs), integrated circuits (ICs), digital circuit elements, analog circuit elements, combinational logic circuits, digital signal processors (DSPs), complex programmable logic devices (CPLDs), etc. may execute the computer readable program instructions/code by utilizing state information of the computer readable program instructions to personalize the electronic circuitry, in order to perform aspects of the present disclosure. Multiple components of the hardware may be integrated, such as on a single die, in a single package, or on a single printed circuit board or logic board. For example, multiple components of the hardware may be implemented as a system-on-chip. A component, or a set of integrated components, may be referred to as a chip, chipset, chiplet, or chip stack. Examples of a system-on-chip include a radio frequency (RF) system-on-chip, an artificial intelligence (AI) system-on-chip, a video processing system-on-chip, an organ-on-chip, a quantum algorithm system-on-chip, etc.

Examples of processing hardware may include a central processing unit (CPU), a graphics processing unit (GPU), an approximate computing processor, a quantum computing processor, a parallel computing processor, a neural network processor, a signal processor, a digital processor, a data processor, an embedded processor, a microprocessor, and a co-processor. The co-processor may provide additional processing functions and/or optimizations, such as for speed or power consumption. Examples of a co-processor include a math co-processor, a graphics co-processor, a communication co-processor, a video co-processor, and an artificial intelligence (AI) co-processor.

In some implementations, the flowchart and block diagrams in the figures illustrate the architecture, functionality, and operation of possible implementations of apparatus (systems), methods and computer program products according to various implementations of the present disclosure. Each block in the flowchart and/or block diagrams, and combinations of blocks in the flowchart and/or block diagrams, may represent a module, segment, or portion of code, which comprises one or more executable computer program instructions for implementing the specified logical function(s)/act(s). These computer program instructions may be provided to a processor of a general purpose computer, special purpose computer, or other programmable data processing apparatus to produce a machine, such that the computer program instructions, which may execute via the processor of the computer or other programmable data processing apparatus, create the ability to implement one or more of the functions/acts specified in the flowchart and/or block diagram block or blocks or combinations thereof. It should be noted that, in some implementations, the functions noted in the block(s) may occur out of the order noted in the figures (or combined or omitted). For example, two blocks shown in succession may, in fact, be executed substantially concurrently, or the blocks may sometimes be executed in the reverse order, depending upon the functionality involved.

In some implementations, these computer program instructions may also be stored in a computer-readable memory that can direct a computer or other programmable data processing apparatus to function in a particular manner, such that the instructions stored in the computer-readable memory produce an article of manufacture including instruction means which implement the function/act specified in the flowchart and/or block diagram block or blocks or combinations thereof.

In some implementations, the computer program instructions may also be loaded onto a computer or other programmable data processing apparatus to cause a series of operational steps to be performed (not necessarily in a particular order) on the computer or other programmable apparatus to produce a computer implemented process such that the instructions which execute on the computer or other programmable apparatus provide steps for implementing the functions/acts (not necessarily in a particular order) specified in the flowchart and/or block diagram block or blocks or combinations thereof.

The terminology used herein is for the purpose of describing particular implementations only and is not intended to be limiting of the disclosure. As used herein, the singular forms “a”, “an” and “the” are intended to include the plural forms as well, unless the context clearly indicates otherwise. As used herein, the phrase “at least one of A, B, and C” should be construed to mean a logical (A OR B OR C), using a non-exclusive logical OR, and should not be construed to mean “at least one of A, at least one of B, and at least one of C.” As another example, the language “at least one of A and B” (and the like) as well as “at least one of A or B” (and the like) should be interpreted as covering only A, only B, or both A and B, unless the context clearly indicates otherwise. It will be further understood that the terms “comprises” and/or “comprising,” when used in this specification, specify the presence of stated features, integers, steps (not necessarily in a particular order), operations, elements, and/or components, but do not preclude the presence or addition of one or more other features, integers, steps (not necessarily in a particular order), operations, elements, components, and/or groups thereof. Example sizes/models/values/ranges can have been given, although examples are not limited to the same.

The terms (and those similar to) “coupled,” “attached,” “connected,” “adjoining,” “transmitting,” “receiving,” “connected,” “engaged,” “coupled,” “adjacent,” “next to,” “on top of,” “above,” “below,” “abutting,” and “disposed,” used herein is to refer to any type of relationship, direct or indirect, between the components in question, and is to apply to electrical, mechanical, fluid, optical, electromagnetic, electromechanical, or other connections. Additionally, the terms “first,” “second,” etc. are used herein only to facilitate discussion, and carry no particular temporal or chronological significance unless otherwise indicated. The terms “cause” or “causing” means to make, force, compel, direct, command, instruct, and/or enable an event or action to occur or at least be in a state where such event or action is to occur, either in a direct or indirect manner. The term “set” does not necessarily exclude the empty set—in other words, in some circumstances a “set” may have zero elements. The term “non-empty set” may be used to indicate exclusion of the empty set—that is, a non-empty set must have one or more elements, but this term need not be specifically used. The term “subset” does not necessarily require a proper subset. In other words, a “subset” of a first set may be coextensive with (equal to) the first set. Further, the term “subset” does not necessarily exclude the empty set—in some circumstances a “subset” may have zero elements.

The corresponding structures, materials, acts, and equivalents (e.g., of all means or step plus function elements) that may be in the claims below are intended to include any structure, material, or act for performing the function in combination with other claimed elements as specifically claimed. While the disclosure describes structures corresponding to claimed elements, those elements do not necessarily invoke a means plus function interpretation unless they explicitly use the signifier “means for.” Unless otherwise indicated, recitations of ranges of values are merely intended to serve as a shorthand way of referring individually to each separate value falling within the range, and each separate value is hereby incorporated into the specification as if it were individually recited. While the drawings divide elements of the disclosure into different functional blocks or action blocks, these divisions are for illustration only. According to the principles of the present disclosure, functionality can be combined in other ways such that some or all functionality from multiple separately-depicted blocks can be implemented in a single functional block; similarly, functionality depicted in a single block may be separated into multiple blocks. Unless explicitly stated as mutually exclusive, features depicted in different drawings can be combined consistent with the principles of the present disclosure.

The description of the present disclosure has been presented for purposes of illustration and description but is not intended to be exhaustive or limited to the disclosure in the form disclosed. After reading the present disclosure, many modifications, variations, substitutions, and any combinations thereof will be apparent to those of ordinary skill in the art without departing from the scope and spirit of the disclosure. The implementation(s) were chosen and described in order to explain the principles of the disclosure and the practical application, and to enable others of ordinary skill in the art to understand the disclosure for various implementation(s) with various modifications and/or any combinations of implementation(s) as are suited to the particular use contemplated. The features of any dependent claim may be combined with the features of any of the independent claims or other dependent claims.

Having thus described the disclosure of the present application in detail and by reference to implementation(s) thereof, it will be apparent that modifications, variations, and any combinations of implementation(s) (including any modifications, variations, substitutions, and combinations thereof) are possible without departing from the scope of the disclosure defined in the appended claims.

What is claimed is:

1. A method for creating a nanomaterial coated, mandrel-coiled twisted and coiled polymer fishing line (TCP_{FL}) actuator comprising:
fabricating a plurality of mandrel-coiled TCP_{FL} muscles, wherein fabricating the plurality of mandrel-coiled TCP_{FL} muscles includes;
synthesizing Graphene-C-Ni-PVA solution; and
coating the TCP_{FL} with the Graphene-C-Ni-PVA solution.

2. The method of claim 1, wherein fabricating the plurality of mandrel-coiled TCP_{FL} muscles further includes twisting of polymer fibers.

3. The method of claim 1, wherein fabricating the plurality of mandrel-coiled TCP_{FL} muscles further includes incorporating a resistance wire into the plurality of mandrel-coiled TCP_{FL} muscles.

4. The method of claim 1, wherein fabricating the plurality of mandrel-coiled TCP_{FL} muscles further includes utilizing a mandrel coiling process.

5. The method of claim 1, wherein fabricating the plurality of mandrel-coiled TCP_{FL} muscles further includes utilizing a thermal annealing process.

6. The method of claim 1, wherein synthesizing the Graphene-C-Ni-PVA solution includes:
dissolving an amount of PVA in water to create a solution;
adding Graphene powder, mesoporous C, and Ni nanoparticles to the solution; and
removing supernatant from the solution.

7. The method of claim 6, wherein coating the TCP_{FL} with the Graphene-C-Ni-PVA solution includes:
placing the TCP_{FL} in the solution;
shaking the TCP_{FL} in the solution;
drying the TCP_{FL} by placing the TCP_{FL} in a pre-heated environment for a predetermined amount of time; and
crimping the TCP_{FL}.

8. A computer program product residing on a computer readable storage medium having a plurality of instructions stored thereon which, when executed across one or more processors, causes at least a portion of the one or more processors to perform operations for creating a nanomaterial coated, mandrel-coiled twisted and coiled polymer fishing line (TCP_{FL}) actuator comprising:
fabricating a plurality of mandrel-coiled TCP_{FL} muscles, wherein fabricating the plurality of mandrel-coiled TCP_{FL} muscles includes;
synthesizing Graphene-C-Ni-PVA solution; and
coating the TCP_{FL} with the Graphene-C-Ni-PVA solution.

9. The computer program product of claim 8, wherein fabricating the plurality of mandrel-coiled TCP_{FL} muscles further includes twisting of polymer fibers.

10. The computer program product of claim 8, wherein fabricating the plurality of mandrel-coiled TCP_{FL} muscles further includes incorporating a resistance wire into the plurality of mandrel-coiled TCP_{FL} muscles.

11. The computer program product of claim 8, wherein fabricating the plurality of mandrel-coiled TCP_{FL} muscles further includes utilizing a mandrel coiling process.

12. The computer program product of claim 8, wherein fabricating the plurality of mandrel-coiled TCP_{FL} muscles further includes utilizing a thermal annealing process.

13. The computer program product of claim 8, wherein synthesizing the Graphene-C-Ni-PVA solution includes:
dissolving an amount of PVA in water to create a solution;
adding Graphene powder, mesoporous C, and Ni nanoparticles to the solution; and
removing supernatant from the solution.

14. The computer program product of claim 13, wherein coating the TCP_{FL} with the Graphene-C-Ni-PVA solution includes:
placing the TCP_{FL} in the solution;
shaking the TCP_{FL} in the solution;
drying the TCP_{FL} by placing the TCP_{FL} in a pre-heated environment for a predetermined amount of time; and
crimping the TCP_{FL}.

15. A nanomaterial coated, mandrel-coiled twisted and coiled polymer fishing line (TCP_{FL}) actuator comprising:
a plurality of mandrel-coiled TCP_{FL} muscles, wherein the plurality of mandrel-coiled TCP_{FL} muscles are fabricated, wherein the plurality of mandrel-coiled TCP_{FL} muscles is coated with a synthesized Graphene-C-Ni-PVA solution.

16. The TCP_{FL} actuator of claim 15, wherein twisting of polymer fibers is utilized to fabricate the plurality of mandrel-coiled TCP_{FL} muscles.

17. The TCP_{FL} actuator of claim 15, wherein the plurality of mandrel-coiled TCP_{FL} muscles includes a resistance wire incorporated into the plurality of mandrel-coiled TCP_{FL} muscles.

18. The TCP_{FL} actuator of claim 15, wherein a mandrel coiling process and a thermal annealing process are utilized to fabricate the plurality of mandrel-coiled TCP_{FL} muscles.

19. The TCP_{FL} actuator of claim 15, wherein the Graphene-C-Ni-PVA solution is synthesized by at least one of:
an amount of PVA dissolved in water to create a solution;
Graphene powder, mesoporous C, and Ni nanoparticles added to the solution; and
supernatant removed from the solution.

20. The TCP_{FL} actuator of claim 19, wherein the TCP_{FL} is coated with the Graphene-C-Ni-PVA solution by at least one of:
the TCP_{FL} placed in the solution;
the TCP_{FL} shaken in the solution;
the TCP_{FL} dried by placing the TCP_{FL} in a pre-heated environment for a predetermined amount of time; and
wherein the TCP_{FL} is crimped.
